(19) United States
(12) Patent Application Publication
(10) Pub. No.: US 2019/0290183 A1
(43) Pub. Date: Sep. 26, 2019

(54) RECOVERY EVALUATION APPARATUS AND NON-TRANSITORY COMPUTER READABLE MEDIUM

(71) Applicant: FUJI XEROX CO., LTD., Tokyo (JP)

(72) Inventors: Takao NAITO, Kanagawa (JP); Shin TAKEUCHI, Kanagawa (JP); Osamu TOCHIKUBO, Kanagawa (JP)

(73) Assignee: FUJI XEROX CO., LTD., Tokyo (JP)

(21) Appl. No.: 16/166,184

(22) Filed: Oct. 22, 2018

(30) Foreign Application Priority Data
Mar. 22, 2018 (JP) 2018-054752

(51) Int. Cl. A61B 5/00; A61B 3/00; A61B 5/103; A61B 5/11

(52) U.S. Cl. CPC A61B 5/4023; A61B 3/0091; A61B 5/1114; A61B 5/1036; A61B 3/0041

(57) ABSTRACT

A recovery evaluation apparatus includes a display that displays an image, an evaluating unit that evaluates balance of a subject who is viewing the image, a controller that, in accordance with temporal variation of an evaluation value of the balance, varies a degree of a visual stimulus provided by the image, and a recovery evaluation unit that evaluates balance recovery, the balance recovery representing balance recovery of the subject upon varying of the degree of the visual stimulus.

### CROSS-REFERENCE TO RELATED APPLICATIONS

This application is based on and claims priority under 35 USC 119 from Japanese Patent Application No. 2018-054752 filed Mar. 22, 2018.

### BACKGROUND

### (i) Technical Field

The present disclosure relates to a recovery evaluation apparatus, and a non-transitory computer readable medium.

### (ii) Related Art

The advent of aging society is creating an urgent need for prevention/early detection of health disorders such as metabolic syndrome, locomotive syndrome, and dementia, which constitute major impediments to extended healthy life expectancy/nursing-care prevention.

Japanese Patent No. 5548267 discloses an apparatus described below. With this apparatus, measurements relating to the performance of a subject, such as stability information, eye movement data, physiological information, or other information, are made both with and without a visual stimulus being provided to the subject. The two sets of collected data are compared to evaluate the subject's ability to visualize the visual stimulus. The apparatus includes a display that provides a visual stimulus to an individual for a first period of time, and at least one stability measurement device that measures the balance of the individual during a second period of time not coextensive with the first period of time and during which the individual is visualizing the visual stimulus.

However, since the balance function recovery of an individual in response to a change in visual stimulus is closely associated with visual cognitive function and locomotive dysfunction, no technique has yet been established to evaluate such balance function recovery.

### SUMMARY

Aspects of non-limiting embodiments of the present disclosure relate to a technique that makes it possible to evaluate the recovery of balance function of an individual in response to a change in visual stimulus.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided a recovery evaluation apparatus including a display that displays an image, an evaluating unit that evaluates balance of a subject who is viewing the image, a controller that, in accordance with temporal variation of an evaluation value of the balance, varies a degree of a visual stimulus provided by the image, and a recovery evaluation unit that evaluates balance recovery, the balance recovery representing balance recovery of the subject upon varying of the degree of the visual stimulus.

### BRIEF DESCRIPTION OF THE DRAWINGS

Exemplary embodiment of the present disclosure will be described in detail based on the following figures, wherein:

FIG. 1 illustrates a general configuration according to an exemplary embodiment;

FIG. 2 is a block diagram illustrating the configuration of an apparatus according to the exemplary embodiment;

FIG. 3 illustrates head oscillations and foot pressure oscillations according to the exemplary embodiment;

FIG. 4 is a graph according to the exemplary embodiment, illustrating temporal variations of visual stimulus and balance;

FIG. 5 is a graph according to the exemplary embodiment, illustrating a case with good balance with respect to visual stimulus;

FIG. 6 is a graph according to the exemplary embodiment, illustrating a case with poor balance with respect to visual stimulus;

FIGS. 7A to 7C are graphs according to the exemplary embodiment, illustrating patterns of balance recovery with respect to visual stimulus;

FIGS. 8A to 8C are graphs illustrating visual stimulus patterns according to the exemplary embodiment;

FIG. 9 is a test process flowchart according to the exemplary embodiment;

FIG. 10 is an overall process flowchart according to the exemplary embodiment;

FIG. 11 is an illustration (No. 1) of how a visual stimulus is varied in accordance with the exemplary embodiment;

FIG. 12 is an illustration (No. 2) of how a visual stimulus is varied in accordance with the exemplary embodiment; and

FIG. 13 is an illustration (No. 3) of how a visual stimulus is varied in accordance with the exemplary embodiment.

### DETAILED DESCRIPTION

An exemplary embodiment of the present disclosure will be described below with reference to the drawings.

The basic principle of the exemplary embodiment is based on the assumption that standing posture balance can be determined by whether markers of individual body segments such as the head and trunk are aligned in a substantially straight line. According to the basic principle, standing posture balance is evaluated by detecting the center of gravity (COG) of the head and the COG of the body (trunk) and then evaluating the relationship between the head COG and the body COG, more specifically, the relationship between the respective positions of the head COG and body COG as projected on the floor surface. If the respective positions of the head COG and body COG projected on the floor surface substantially coincide with each other, it can be said that the standing posture is balanced or the standing posture is correct. If the respective positions of the head COG and body COG projected on the floor surface deviate from each other beyond an allowable range, it can be said that the standing posture is unbalanced or the standing posture is abnormal. In this regard, the expression “substantially coincide” means that some tolerance is permitted to allow for differences between individuals and statistical errors.

The head COG position (position projected on the floor surface) can be detected from, for example, an image of a subject in standing posture captured with a camera located above the subject's head. The body COG position (position projected on the floor surface) can be detected by using, for example, a signal obtained from a foot pressure (body pressure) sensor on which the subject steps in standing posture. The head COG position represents the COG position of only the head, and the body COG position represents the COG position of the whole body including the head.

If the two COG positions coincide, this means that the COG of the head and the COG of the body are aligned in substantially the same vertical line, and hence the standing posture can be evaluated as being balanced. If the two COG positions deviate from each other, the degree or temporal variation of this deviation can be used to quantitatively evaluate the degree of deterioration in standing posture balance. As described above, according to the exemplary embodiment, rather than using only the COG position of the body, both the head COG position and the body COG position are used, and the head COG position and the body COG position are associated with each other to evaluate the balance of the subject in standing posture.

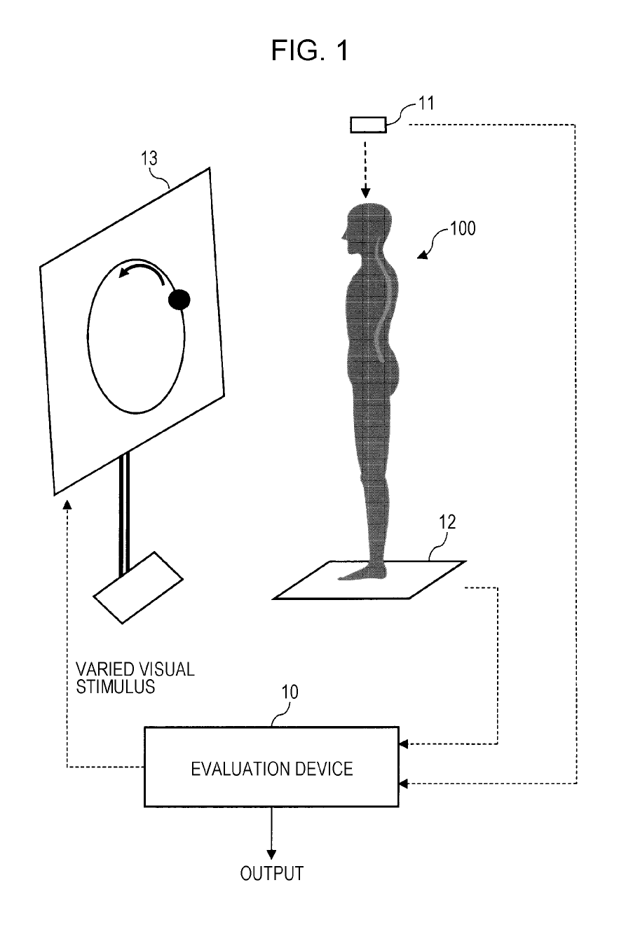

FIG. 1 illustrates a general configuration of a recovery evaluation apparatus according to the exemplary embodiment.

A recovery evaluation apparatus presents a subject 100 with a visual stimulus, which means a stimulus to the visual sense. The recovery evaluation apparatus detects the head oscillations and foot pressure oscillations of the subject 100 in this state to evaluate the balance function of the subject 100. In particular, the recovery evaluation apparatus evaluates the recovery of the subject's balance function upon varying of the visual stimulus.

The recovery evaluation apparatus includes an evaluation device 10, a head sensor 11, a foot pressure sensor 12, and a screen 13.

The head sensor 11 is disposed directly above the subject to detect the location of the head of the subject 100. The head sensor 11 outputs the detected head location data to the evaluation device 10.

The foot pressure sensor 12 detects the foot pressure of the subject 100. The foot pressure sensor 12 outputs the detected foot pressure data to the evaluation device 10.

The screen 13 is disposed in front of the subject 100. The screen 13 displays various images to present a visual stimulus to the subject 100. Instead of the screen 13, a head mount display or eyeglasses worn by the subject 100 may be used to provide a visual stimulus based on virtual reality (VR) technologies.

The evaluation device 10 detects the head oscillations and foot pressure oscillations of the subject 100 who views various patterns displayed on the screen 13 while standing directly below the head sensor 11 and stepping on the foot pressure sensor 12 in standing posture. The evaluation device 10 uses the detected head oscillations and the detected foot pressure oscillations to evaluate the balance function of the subject 100, more specifically, the visual cognitive function and locomotive dysfunction of the subject 100. At this time, in accordance with the degree of balance function of the subject 100, the evaluation device 10 adaptively varies the image displayed on the screen 13 to thereby vary the degree of the visual stimulus being presented. The evaluation device 10 then evaluates the recovery of the balance function of the subject 100 with respect to variation of the presented visual stimulus. The evaluation device 10 outputs the evaluation results to the display or other devices for presentation to the subject 100 or to the concerned medical personnel.

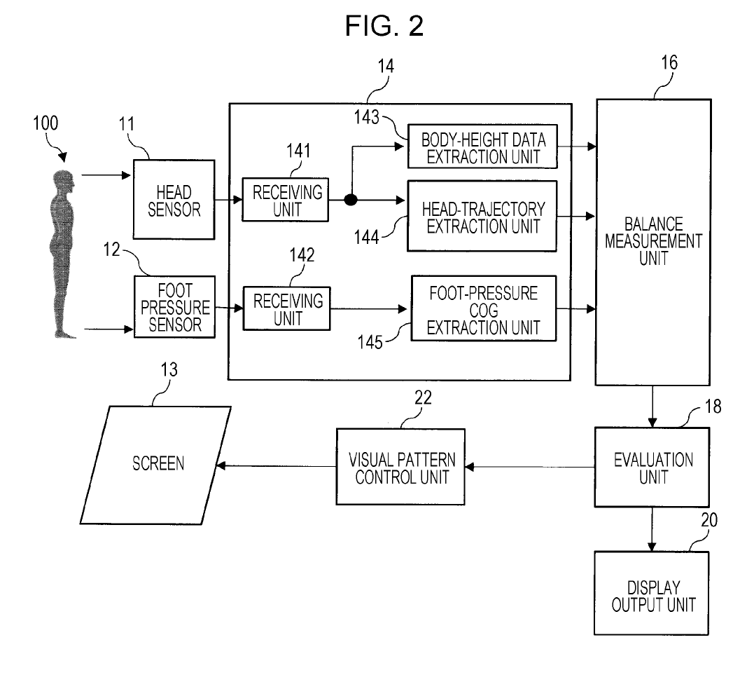

FIG. 2 is a block diagram illustrating the configuration of the recovery evaluation apparatus according to the exemplary embodiment.

The head sensor 11 is implemented by, for example, a 3D camera or an optical sensor. The head sensor 11 detects the location of the head of the subject 100. For example, the head sensor 11 is mounted facing downward on a support extended horizontally from an upper portion of a supporting post. The head sensor 11 is mounted on the support such that the head sensor 11 is located substantially over the subject's head when the subject is in standing position with the feet placed on a predetermined location on the foot pressure sensor 12. If a 3D camera is used as the head sensor 11, an image of the subject is captured from above the subject's head, and the obtained image data is output. Desirably, the support is able to move up and down along the supporting post, thus allowing the distance between the subject's head and the 3D camera to be adjusted in accordance with the subject's body height. Typically, a 3D camera is used to capture 3D content for display on a 3D display, and constructed as a combination of two cameras to enable capture of the image for the right eye and the image for the left eye. The two cameras are arranged horizontally such that their relative position is close to the relative position of human eyes, with the distance between their respective lenses being set to less than or equal to 50 mm to match the spacing between human eyes. Two cameras may be integrated for use as a 3D camera. For example, with a lens and an imaging device forming a pair, the 3D may be made up of two such pairs, or may be a combination of an imaging device with the lens for the right eye and the lens for the left eye. In this case, the imaging device is divided into two regions, one for the lens for the right eye and one for the lens for the left eye, thus enabling simultaneous capture of both the image for the right eye and the image for the left eye. The 3D camera may be used to detect the distances to various parts of the subject's head. The head sensor 11 outputs the detected head location data to a measurement unit 14.

The foot pressure sensor (body pressure sensor) 12 detects the foot pressure of the subject 100 in standing posture. The foot pressure sensor 12 is placed on top of a footboard to detect the subject's foot pressure. Left and right human footprints are marked on the foot pressure sensor 12. The subject places his or her feet on the foot pressure sensor 12 with each footprint mark used as a reference position. The positional relationship between the head sensor 11 and the foot pressure sensor 12, more specifically, the positional relationship between the head sensor 11 and the footprint marks on the foot pressure sensor 12 is determined such that the head sensor 11 is located over the subject's head when the subject is in standing position with the feet placed on the footprint marks. The foot pressure sensor 12 is implemented by, for example, a pressure sensor such as a piezoelectric element. The foot pressure sensor 12 converts the pressure (load) applied upon placing of the subject's feet on the foot pressure sensor 12 into an electrical signal, and outputs the resulting electrical signal. The foot pressure sensor 12 may be placed across the entire area of each footprint mark, or may be placed only at a specific location on each footprint mark. For example, the foot pressure sensor 12 may be placed at three locations (a total of six locations for both the left and right footprints), one near the base of the big toe, one near the base of the little toe, and one at the heel. The foot pressure (load) detected by the foot pressure sensor 12 is used to calculate the COG position of the subject's body, and thus the foot pressure sensor 12 may be placed at any location suited for this purpose. The foot pressure sensor 12 outputs the detected foot pressure data to the measurement unit 14.

The measurement unit 14 includes receiving units 141 and 142, a body-height data extraction unit 143, a head-trajectory extraction unit 144, and a foot-pressure COG extraction unit 145.

The receiving unit 141 receives head location data from the head sensor 11, and outputs the received head location data to the body-height data extraction unit 143 and the head-trajectory extraction unit 144.

The receiving unit 142 receives foot pressure data from the foot pressure sensor 12, and outputs the received foot pressure data to the foot-pressure COG extraction unit 145.

The body-height data extraction unit 143 extracts the subject's body height data by use of head location data obtained from the head sensor 11. For example, by using a 3D camera as the head sensor 11, the subject's body height data is extracted based on the difference between the shortest distance from the 3D camera (corresponding to the top of the head) and the longest distance from the 3D camera (corresponding to the floor surface). This distance measurement may be performed by using a laser measurement instrument or other devices. The body height data may be used to calculate a conversion factor k used to convert the subject's body height into a reference body height to perform scale adjustment.

The head-trajectory extraction unit 144 receives head location data obtained by the head sensor 11, and calculates the COG position g_{head} the subject's head from the received data. More precisely, the head-trajectory extraction unit 144 calculates the head COG position as projected on the floor surface (surface in contact with the feet). Then, the head-trajectory extraction unit 144 extracts a trajectory representing how the calculated head location varies with time.

The head COG position g_{head} may be calculated by having the subject 100 wear a headset (head marker) with a protruding marker, and determining the vertex of the head marker as the head COG position g_{head}.

The foot-pressure COG extraction unit 145 receives foot pressure data obtained from the foot pressure sensor 12, and calculates the COG position g_{fp} of the subject's body from the obtained data. More precisely, the foot-pressure COG extraction unit 145 calculates the body COG position as projected on the floor surface (surface in contact with the feet). Techniques for calculating the COG position of a person when the person steps on a pressure sensor are known in the art. For example, if a pressure sensor is placed at each of three locations (a total of six locations for both the left and right footprints), one near the base of the big toe, one near the base of the little toe, and one at the heel, electrical signals from a total of six such pressure sensors are processed to calculate a pressure distribution, and the center of the pressure distribution is determined as the body COG position.

A balance measurement unit 16 measures the subject's balance by use of the following pieces of data obtained by the measurement unit 14: the subject's body height data, the trajectory (Lissajous) of the head COG position, and the trajectory (Lissajous) of the body COG position. If the respective positions of the head COG and body COG projected on the floor surface substantially coincide with each other, it can be said that the standing posture is balanced or the standing posture is correct. If the respective positions g_{head} and g_{fp} of the head COG and body COG projected on the floor surface deviate from each other beyond an allowable range, it can be said that the standing posture is unbalanced or the standing posture is abnormal. In this regard, the expression “substantially coincide” means that some tolerance is permitted to allow for differences between individuals and statistical errors. Under the basic principle mentioned above, the balance measurement unit 16 quantities and measures the subject's overall standing posture balance based on the deviation between the head COG position g_{head} and the body COG position g_{fp} and the results of Lissajous analysis of each COG position.

In calculating the head COG position g_{head} and the body COG position g_{fp}, the balance measurement unit 16 executes other data processing required. Examples of such data processing required include scale adjustment, positional adjustment, noise cutting, and real distance conversion.

In a scale adjustment process, by taking the subject's body height into account, the conversion factor k for converting the subject's body height into a reference body height is calculated.

In a positional adjustment process, if the center positions of the head sensor 11 and foot pressure sensor 12 are misaligned, the two positions are adjusted to align with each other. Letting the amount of misalignment between the head sensor 11 and the foot pressure sensor 12 be (mx, my), the value (mx, my) is used to as an offset to correct for misalignment.

In a noise cutting process, abrupt changes are removed as noise. Specifically, letting Th be a threshold, Lₓ be a data value obtained at a given time instant, and Lₓ₋₁ be a data value obtained at the immediately preceding time instant. In this case, if Lₓ−Lₓ₋₁ exceeds the threshold Th, Lₓ is replaced by Lₓ₋₁ to thereby remove noise.

In a real distance conversion process, the distance between pixels on the image obtained by the head sensor 11 is converted into a real distance. For example, the distance equivalent to several pixels in the image obtained with the 3D camera is converted into 1 cm. For example, such a process is performed for a case in which the reference body height is 165 cm, and the subject's body height is 175 cm. Assuming that the distance (reference distance) SL between the head sensor 11 and a subject with a reference body height is equal to 500 mm, then for a subject with a body height of 175 cm, the distance SL between the head sensor 11 and the subject is equal to 400 mm. If the distance from the head sensor 11 changes as described above, the Lissajous size changes even when the actual movement of the subject is the same. That is, the smaller the distance from the head sensor 11, the greater the Lissajous size for the same amount of movement. Accordingly, the Lissajous in the evaluation plane for a subject with a body height of 175 cm needs to be converted into the Lissajous in the evaluation plane for a subject with the reference body height of 165 cm. The conversion factor k is given by:

k=distance from head sensor 11/reference distance.

Accordingly, letting the coordinates of the origin of the evaluation plane be (0, 0), the upper-right coordinates be (640, 480), and the center coordinates be (320, 240), coordinates (x, y) in the evaluation plane are converted into coordinates (x′, y′) in the evaluation plane as follows.

x′=320+k·(x−320)

y′=240+k·(y−240).

If the above-mentioned misalignment (mx, my) is also taken into account to compensate for the misalignment, the resulting coordinates (x′, y′) are obtained as follows.

x′=320+k·(x−320)+mx

y′=240+k·(y−240)+my

The balance measurement unit 16 quantitatively evaluates the subject's standing posture based on Lissajous analysis. Letting hd be the movement distance of the head COG position g_{head}, be the area of its Lissajous figure, fd be the movement distance of the body COG position g_{fp}, and fa be the area of its Lissajous figure, the subject's standing posture is evaluated by using these values selectively or in combination. For example, the standing posture is evaluated by using the following balance measurement value:

balance measurement value=(fd+hd).

Alternatively, the standing posture is evaluated by using the following balance measurement value:

balance measurement value=(fd+hd+fa+ha).

In this regard, for the balance measurement value, a smaller numerical value can be evaluated as representing better balance. Alternatively, the balance measurement value may be evaluated by using the distance d_{gg} between the head COG position g_{head} and body COG position g_{fp}. For example, the balance measurement value may be obtained as follows:

balance measurement value=(d_{gg}+fd+hd+fa+ha).

The balance measurement unit 16 outputs the calculated balance measurement value to an evaluation unit 18.

The evaluation unit 18 functions as an evaluating unit that evaluates the balance of the subject 100 who is being presented with a visual stimulus, and as a recovery evaluation unit that evaluates the balance recovery of the subject 100 upon varying of the degree of the visual stimulus being presented. From the balance measurement value calculated by the balance measurement unit 16, and the level of the visual stimulus presented to the subject 100 by using the screen 13, the evaluation unit 18 quantitatively evaluates the overall balance of the subject 100 with respect to the visual stimulus, including visual cognitive function and the degree of locomotive dysfunction. Specifically, the evaluation unit 18 calculates a visual balance score as follows as an evaluation value of the balance of the subject 100:

visual balance score=Σ(visual stimulus level*1/balance measurement value).

The visual stimulus is evaluated on a scale of, for example, five levels from Level 0 to Level 4, with a predetermined coefficient assigned to each level. For example, the following coefficients are assigned to individual levels.

Level 0: coefficient=1.0

Level 1: coefficient=1.2

Level 2: coefficient=1.4

Level 3: coefficient=1.6

Level 4: coefficient=2.0

As the balance measurement value, the balance measurement value calculated by the balance measurement unit 16 is used. Since a smaller balance measurement value indicates better balance, it follows that for the visual balance score, a higher value indicates better balance function. The evaluation unit 18 outputs the evaluation results to a display output unit 20, and also to a visual pattern control unit 22.

The visual pattern control unit 22 functions as a controller that, in accordance with the temporal variation of the balance evaluation value, varies the degree of a visual stimulus provided by an image. The visual pattern control unit 22 varies, in accordance with the evaluation results from the evaluation unit 18, a visual pattern displayed as an image on the screen 13 to thereby vary the level of the visual stimulus presented to the subject 100. Examples of varying of a visual pattern include changing a currently displayed image to another image, and changing the manner of display of a currently displayed image. Examples of changing the manner of display of an image at this time include changing the shape of the image, changing the size of the image, changing the angle of the image, rotating the image, flashing the image, and highlighting the image. The visual pattern control unit 22 sequentially increases the visual stimulus level in accordance with the evaluation results obtained from the evaluation unit 18, and the temporal variation of the visual balance score of the subject 100 at this time is evaluated by the evaluation unit 18. Feeding back the visual balance score to the visual stimulus level in this way makes it possible to present the subject 100 with an optimum level of visual stimulus according to the visual balance score.

The measurement unit 14, the balance measurement unit 16, the evaluation unit 18, the visual pattern control unit 22, and the display output unit 20 may be implemented by a computer including a processor, a memory, an input/output interface, a communication interface, and a display. The processor reads and executes a program stored in a ROM, an HDD, an SSD, or other storage devices to thereby implement the measurement unit 14, the balance measurement unit 16, the evaluation unit 18, and the visual pattern control unit 22. Some functions may be implemented not by software processing performed by execution of the program but by hardware processing. Hardware processing may be performed by using, for example, a circuit such as an ASIC or a field programmable gate array (FPGA).

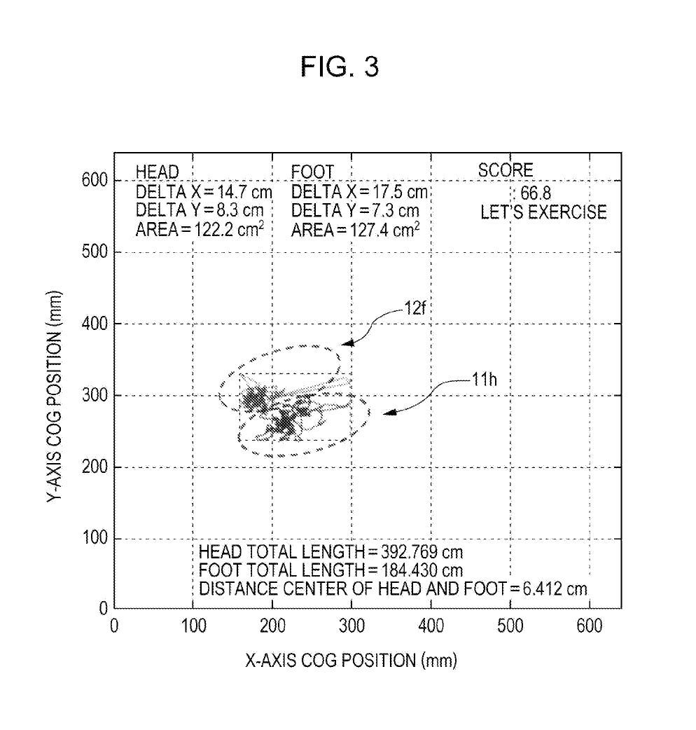

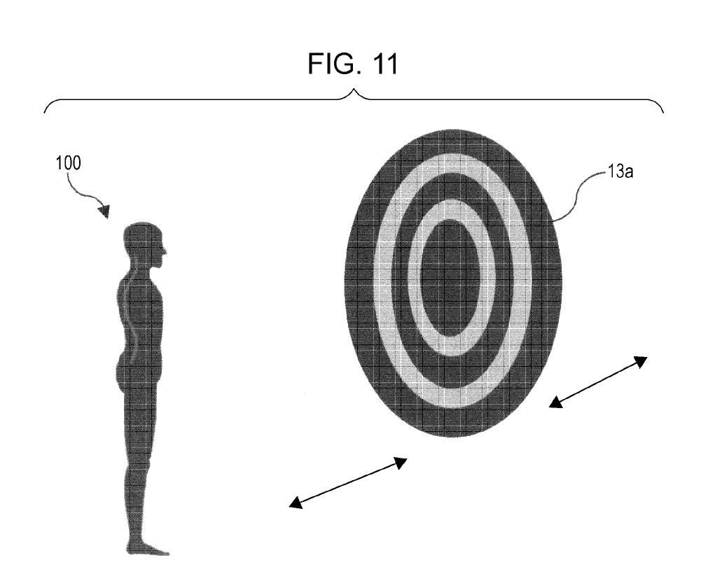

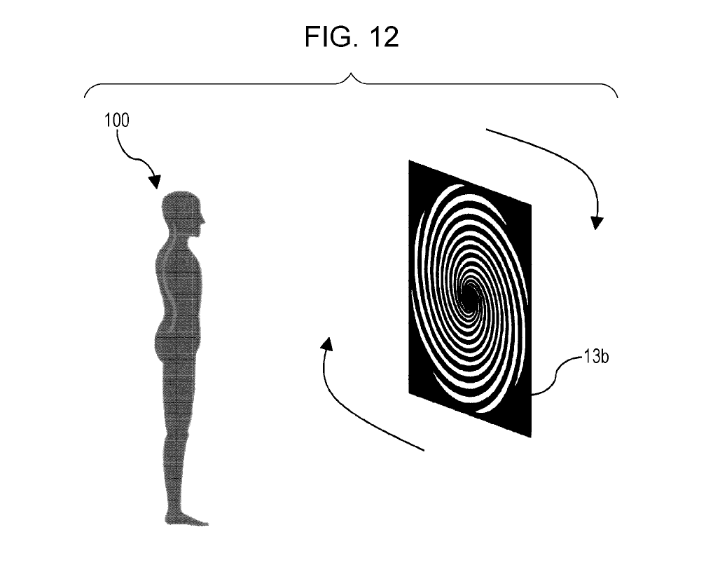

FIG. 3 illustrates an exemplary head COG position g_{head} and an exemplary body COG position g_{fp} that are measured by the balance measurement unit 16. The horizontal axis represents a given direction (X-axis) on the floor surface serving as a projection plane, and the vertical axis represents a direction (Y-axis) perpendicular to the X-axis direction. When the subject 100 is in standing posture, the subject's body can be constantly subject to micro-movements or large oscillations. The body COG position g_{fp} can thus fluctuate with the passage of time. Consequently, the body COG position g_{fp} traces out a Lissajous figure. Likewise, the head COG position g_{head} also traces out a Lissajous figure. When the subject 100 is presented with a visual stimulus, the Lissajous figures of the body COG position g_{fp} and head COG position g_{head} can vary with the cognitive function and the degree of locomotive dysfunction of the subject 100. FIG. 3 depicts a Lissajous figure 11f traced by the time series data of the head COG position g_{head}, and a Lissajous figure 12f traced by the time series data of the body COG position g_{fp}.

In correct standing posture (the standing posture of a subject in heathy condition), the head COG position g_{head} and the body COG position g_{fp} substantially coincide. However, when the standing posture goes off balance due to causes such as decreased cognitive function or locomotive dysfunction, the head COG position g_{head} and the body COG position g_{fp} tend to gradually deviate from each other, and their respective Lissajous figures 11h and 12f tend to increase in trajectory length or area. The balance measurement unit 16 calculates a balance measurement value by using, for example, the deviation between the head COG position g_{head} and the body COG position g_{fp} and the trajectory length or area of each of the Lissajous figures 11h and 12f upon presentation of a visual stimulus to the subject.

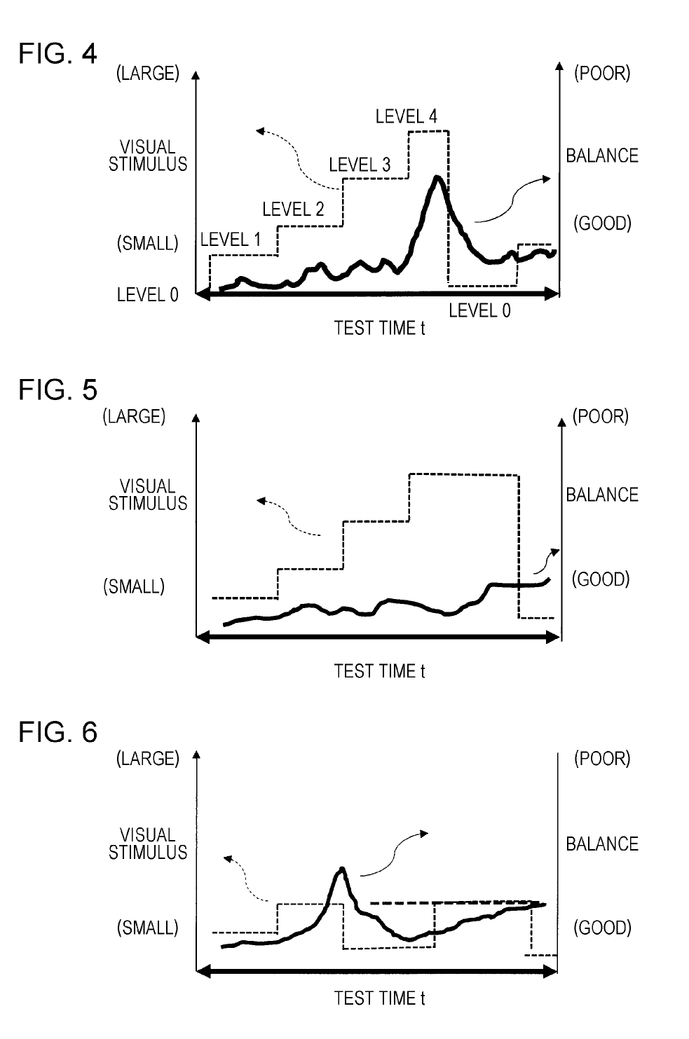

FIG. 4 illustrates the relationship between visual stimulus and balance. In FIG. 4, the horizontal axis represents time, the left vertical axis represents visual stimulus level, and the right horizontal axis represents balance (visual balance score). According to the above-mentioned definition of the visual balance score, a greater numerical value of visual balance score indicates better balance. Accordingly, a higher position along the right vertical axis indicates a smaller visual balance score and hence poorer balance, whereas a lower position along the right vertical axis indicates a greater visual balance score and hence better balance.

With the initial visual stimulus level set to the lowest level, Level 0, the visual stimulus level is increased stepwise as Level 1→Level 2→Level 3→Level 4, and the visual balance scores at individual visual stimulus levels are evaluated by the evaluation unit 18. Generally, as the level of visual stimulus increases, balance deteriorates. In this regard, when balance deteriorates too abruptly, it is not desirable from the safety viewpoint to further increase the level of the visual stimulus presented.

Accordingly, with focus on temporal variation of the visual balance score, the absolute value of the temporal variation of the visual balance score is taken as ΔB. If ΔB exceeds a threshold, the visual stimulus level is not increased but conversely decreased. For example, as illustrated in FIG. 4, when the absolute value ΔB of the temporal variation of the visual balance score exceeds this threshold upon increasing of the visual stimulus level to Level 4, the visual stimulus level is decreased from Level 4 to Level 0, and then gradually increased again from Level 0.

Specifically, the evaluation unit 18 calculates the absolute value ΔB of the temporal variation of the visual balance score and compares the calculated value with a threshold to determine which one is greater than the other. If the absolute value ΔB exceeds the threshold, the evaluation unit 18 outputs the absolute value ΔB to the visual pattern control unit 22. In accordance with this evaluation result from the evaluation unit 18, the visual pattern control unit 22 varies the visual pattern displayed on the screen 13 to decrease the visual stimulus level. In this case, the relationship between a visual pattern displayed on the screen 13, and the degree of the visual stimulus presented by the visual pattern is determined by an experiment or other methods and stored into a memory as a table in advance. By referring to the table in accordance with the evaluation result obtained from the evaluation unit 18, a visual pattern with a degree of visual stimulus appropriate for the evaluation result is selected and displayed on the screen 13. Visual patterns and the degrees of visual stimuli presented by the visual patterns will be described later in further detail.

FIGS. 5 and 6 each illustrate how the visual balance score varies with variation of the visual stimulus pattern.

FIG. 5 represents a case with good visual balance score with respect to variation of the visual stimulus pattern, that is, a case in which the balance function of the subject is relatively good.

If the visual balance score does not deteriorate greatly and the absolute value ΔB of the temporal variation of the visual balance score does not exceed a threshold as the visual stimulus level is sequentially increased from Level 0 to Level 4, the test is ended after the visual stimulus level is increased to Level 4. In this case, the test may be repeated with the visual stimulus level returned to Level 0 again.

FIG. 6 represents a case with poor visual balance score with respect to variation of the visual stimulus pattern, that is, a case in which the balance function of the subject 100 is relatively poor.

In this case, after increasing of the visual stimulus level from Level 0 to Level 1, at the instant when the visual stimulus level is further increased from Level 1 to Level 2, the visual balance score deteriorates and the absolute value ΔB of the temporal variation of the visual balance score exceeds a threshold. Accordingly, the visual stimulus level is decreased from Level 2 to the lowest level, Level 0. Then, for the duration of the test period, Level 2 is set as the upper limit of the visual stimulus level, and the visual balance score is calculated without increasing the visual stimulus level to Level 3 or Level 4.

As described above, if the visual balance score, which is denoted as B, abruptly deteriorates and the absolute value ΔB of the temporal variation of the visual balance score exceeds a threshold, the visual stimulus level is decreased rather than being increased. In this case, there are several possible patterns for how to subsequently vary the visual stimulus level.

In this case, in the exemplary embodiment, the visual stimulus level is varied in accordance with how the visual balance score B of the subject 100 varies with time after the visual stimulus level is decreased to Level 0 as a result of a threshold being exceeded by the absolute value ΔB of the temporal variation of the visual balance score B. In other words, the visual stimulus level is varied in accordance with the recovery of the balance of the subject 100 after the visual stimulus level is decreased to Level 0.

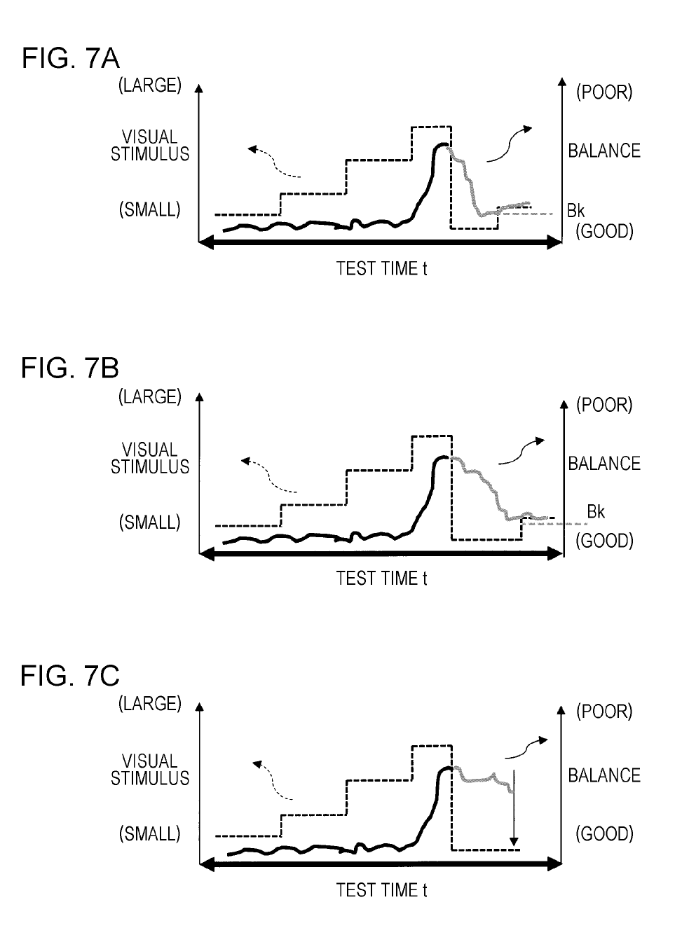

FIGS. 7A to 7C each illustrate how the visual stimulus level is varied in accordance with the balance recovery of the subject 100.

FIG. 7A illustrates a case in which, after the absolute value ΔB of the temporal variation of the visual balance score B exceeds a threshold and the visual stimulus level is decreased to Level 0 as a result, the visual balance score B quickly recovers to a good value. In this case, it is regarded that the deterioration in balance is temporary. Accordingly, the visual stimulus level is quickly increased to Level 1 again after being decreased to Level 0.

More specifically, a reference value Bk, and a first predetermined time T1 and a second predetermined time T2 (T1<T2) are set. If the visual balance score recovers to Bk within the first predetermined time T1, it is regarded that the visual balance score B has quickly recovered to a good value. Accordingly, after the visual stimulus level is decreased to Level 0, the visual stimulus level is quickly increased to Level 1 again.

FIG. 7B illustrates a case in which, after the absolute value ΔB of the temporal variation of the visual balance score B exceeds a threshold and the visual stimulus level is decreased to Level 0 as a result, the visual balance score B recovers to a good value relatively slowly. In this case, it is waited for the visual balance score B to recover to the reference value Bₖ. After the visual balance score B recovers to Bₖ, the visual stimulus level is increased from Level 0 to Level 1 again after a predetermined interval of time t₀.

More specifically, if the visual balance score B does not recover to the reference value Bₖ within the first predetermined time T1 but recovers to the reference value Bₖ within the second predetermined time T2, the visual stimulus level is increased from Level 0 to Level 1 again after the predetermined interval of time t₀.

FIG. 7C illustrates a case in which, after the absolute value ΔB of the temporal variation of the visual balance score B exceeds a threshold and the visual stimulus level is decreased to Level 0 as a result, the visual balance score B does not readily recover to the reference value Bₖ. More specifically, this represents a case in which the visual balance score B does not recover to Bₖ neither within the first predetermined time T1 nor within the second predetermined time T2. In this case, it is regarded that the ability of the subject 100 to recover balance has severely declined, and hence further continuation of the test may be potentially dangerous. Accordingly, the test is promptly stopped without increasing the visual stimulus level from Level 0.

Whether the visual balance score B does not readily recover to a good value may be determined by comparison between the relative magnitudes of a threshold and the absolute value ΔB of the temporal variation of the visual balance score B observed after the visual stimulus level is decreased to Level 0.

The visual stimulus level may be increased sequentially as long as the absolute value ΔB of the temporal variation of the visual balance score B does not exceed a threshold. In this case, the pattern of increasing the visual stimulus level may be varied in accordance with the temporal variation of the visual balance score B of the subject 100.

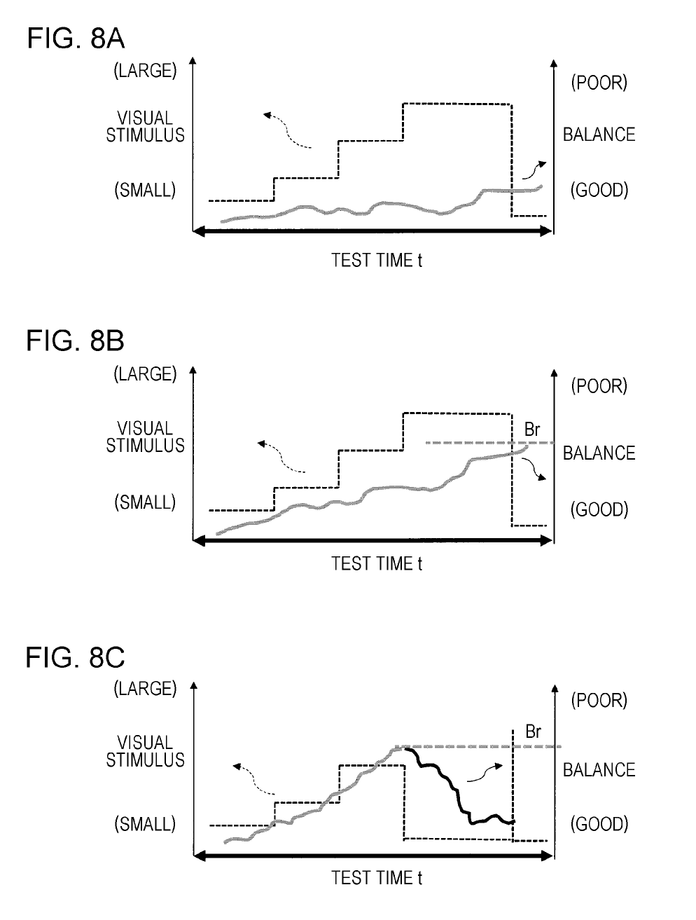

FIGS. 8A to 8C each illustrate how the visual stimulus level is varied in accordance with the temporal variation of the balance function of the subject 100.

FIG. 8A represents a case with in which the relative degree of deterioration in the visual balance score B of the subject 100 is small. In this case, the visual stimulus level is increased from Level 0 to Level 1, Level 2, Level 3, and then Level 4 sequentially at predetermined time intervals.

FIG. 8B represents a case in which the relative degree of deterioration in the visual balance score B of the subject 100 is medium. In this case, the visual stimulus level is increased from Level 0 to Level 4 sequentially at predetermined time intervals as long as the visual balance score B does not exceed an allowable value Br. However, if the visual balance score B deteriorates beyond the allowable value Br, this is regarded as indicating that some abnormality (such as a visual cognitive function abnormality, a locomotive abnormality, or a metal state abnormality) has occurred in the subject 100 and hence further visual stimulus presentation is potentially dangerous. Accordingly, the visual stimulus level is decreased to Level 0.

FIG. 8C represents a case in which the relative degree of deterioration in the visual balance score B of the subject 100 is large. In this case as well, if the visual balance score B exceeds the allowable value Br, this is regarded as indicating that some abnormality has occurred in the subject 100 and hence further visual stimulus presentation is potentially dangerous. Accordingly, the visual stimulus level is decreased to Level 0.

In the case of FIG. 8C, in particular, the absolute value ΔB of the temporal variation of the visual balance score B can exceed a threshold in some situations. Accordingly, the visual stimulus level may be decreased to Level 0 if the visual balance score B exceeds the allowable value Br or if the absolute value ΔB of the temporal variation of the visual balance score B exceeds a threshold.

As illustrated in FIGS. 7A to 8C, in the exemplary embodiment, the visual stimulus level is varied in accordance with the temporal variation of the visual balance score B of the subject 100 to thereby evaluate the balance and balance recovery of the subject 100. Balance recovery is evaluated directly based on whether the visual balance score B temporarily lowered to Level 0 recovers to the reference value Bₖ within the second predetermined time T2 as illustrated in FIGS. 7A to 7C. At the same time, balance recovery may be also evaluated based on how much the visual balance score deteriorates upon increasing of the visual stimulus level. In this sense, it can be said that balance recovery represents balance tolerance or stress tolerance to changing visual stimulus level. If the visual balance score does not exceed a threshold, the visual stimulus level is increased sequentially. Consequently, balance recovery may be calculated at an early stage as compared to if the visual stimulus level is not varied. This makes it possible to shorten the test period required.

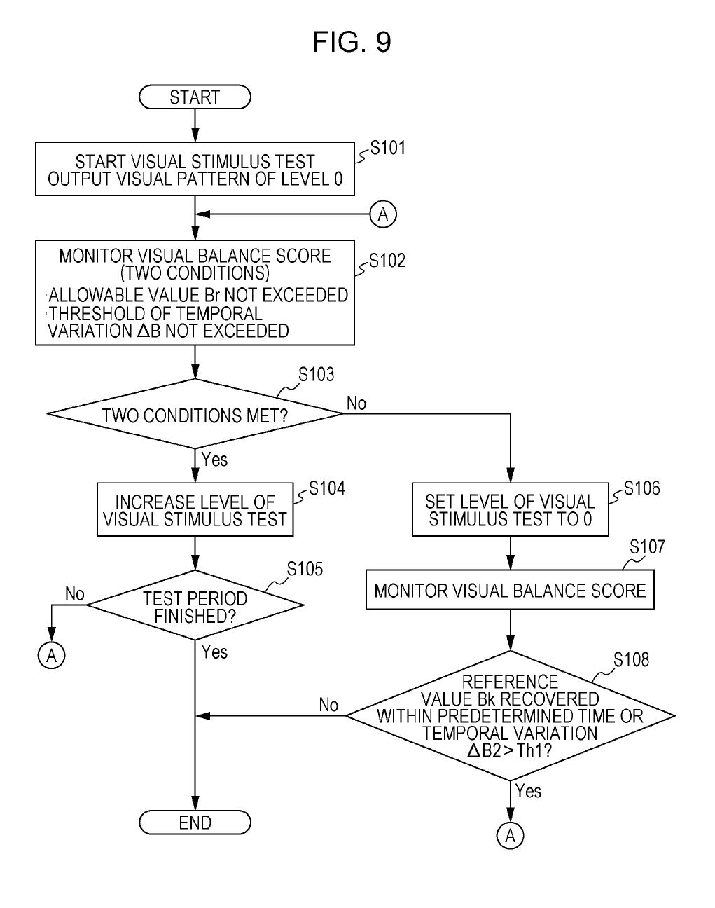

FIG. 9 is a process flowchart according to the exemplary embodiment.

The subject 100 places his or her feet on the footprints on the foot pressure sensor 12, and keeps his or her standing posture while facing the screen 13 located in front of the subject 100. After checking that the subject 100 is ready, the evaluation unit 18 informs the subject 100 of the start of a test by displaying, for example, the following message on the display output unit 20:

“Measurement now begins. Please watch the screen in front of you”.

As the test begins, the visual pattern control unit 22 outputs a visual pattern of the lowest level, Level 0, to the screen 13 (S101). The head sensor 11 detects the location of the head of the subject 100, and outputs the detected head location. The foot pressure sensor 12 detects the foot pressure of the subject 100, and outputs the detected foot pressure. The balance measurement unit 16 calculates a balance measurement value by using the respective Lissajous figures of the head COG position g_{head} and body COG position g_{fp}.

Next, the evaluation unit 18 calculates the visual balance score B with the subject 100 being presented with a visual stimulus of Level 0 (S102). At this time, for example, the evaluation unit 18 uses the balance measurement value calculated by the balance measurement unit 16 to calculate the visual balance score B as follows:

visual balance score=Σ(visual stimulus level*1/balance measurement value).

The visual balance score may be calculated as the inverse of the above-mentioned value. That is, the visual balance score may be calculated as follows:

visual balance score=1/Σ(visual stimulus level*1/balance measurement value).

In this case, a greater numerical value indicates greater deterioration in balance function.

In calculating the visual balance score, the evaluation unit 18 may exclude data obtained within a predetermined time after the start of the measurement. This is because within a predetermined time after the start of the measurement, it is likely that the standing posture of the subject 100 does not stabilize and hence the reliability of the resulting data is low. This predetermined time may be set to any amount of time, for example, two or three seconds.

Further, by using the calculated visual balance score B, the evaluation unit 18 determines whether the following two conditions are met: the value of the visual balance score B does not exceed the allowable value Br, and the absolute value ΔB of the temporal variation of the visual balance score does not exceed a threshold (S102 and S103).

If the visual balance score B does not exceed the allowable value Br, and if the absolute value ΔB of the temporal variation of the visual balance score B does not exceed a threshold (S103: YES), the evaluation unit 18 determines that no particular abnormality has occurred in the subject 100, and outputs the evaluation result to the visual pattern control unit 22.

The visual pattern control unit 22 varies the visual pattern in accordance with the evaluation result from the evaluation unit 18 so that the visual stimulus presented by the pattern is increased by one level (S104). That is, if the current level is Level 0, the level is increased to Level 1, and if the current level is Level 1, the level is increased to Level 2. Then, it is determined whether the test period is finished (S105). If the test period is not finished, the procedure from 5102 onward is repeated (S105: NO). This test period is determined by the duration of time or by the upper limit of the stimulus level. For example, the test period is finished when the visual balance score is calculated after the visual stimulus level is increased to Level 4. Alternatively, the test period is finished upon elapse of a predetermined time, for example, 20 minutes after the start of the test. After the test period is finished, the evaluation unit 18 displays, on the display output unit 20, the visual balance score calculated up to that point. For example, the following message is output:

“The test is finished. Your score is 80”.

By contrast, if the visual balance score B exceeds the allowable value Br, or if its temporal variation ΔB exceeds a threshold (S103: NO), the evaluation unit 18 determines that an abnormality has occurred in the subject 100, and outputs the obtained result to the visual pattern control unit 22. The visual pattern control unit 22 varies the visual pattern in accordance with the result obtained from the evaluation unit 18 to thereby decrease the visual stimulus level to Level 0 (S106).

Subsequently, the evaluation unit 18 evaluates the visual balance score calculated after the visual stimulus level is decreased to Level 0 (S107). Then, the evaluation unit 18 determines whether the visual balance score B calculated after decreasing of the visual stimulus level to Level 0 has recovered to the reference value Bₖ within the predetermined time T2, or whether its temporal variation ΔB2 has exceeded a threshold Th1 (S108).

If the visual balance score B calculated after decreasing of the visual stimulus level to Level 0 has recovered to the reference value Bₖ within the predetermined time T2, or if its temporal variation ΔB2 has exceeded the threshold Th1 (S108: YES), the evaluation unit 18 determines that there is no problem with the balance recovery of the subject 100, more specifically that the observed balance recovery corresponds to either the case illustrated in FIG. 8A or the case illustrated in FIG. 8B. The evaluation unit 18 then repeats the procedure from S102 onward, and sequentially increases the visual stimulus level from Level 0. Then, after the test period is finished, the evaluation unit 18 outputs the calculated visual balance score. For example, the following message is output:

“The test is finished. Your score is 60. Your balance recovery is normal”.

If the visual balance score B calculated after decreasing of the visual stimulus level to Level 0 has not recovered to the reference value Bₖ within the predetermined time T2, and if the temporal variation ΔB2 of the visual balance score B has not exceeded the threshold Th1 (S108: NO), the evaluation unit 18 determines that there is a problem with the balance recovery of the subject 100, more specifically that the observed balance recovery corresponds to the case illustrated in FIG. 8C. Accordingly, the evaluation unit 18 stops the test without repeating the procedure from S102 onward. Then, after the test is stopped, the evaluation unit 18 outputs the visual balance score calculated up to that point. For example, the following message is output:

“The test has been stopped. Your score is 30. Your balance recovery is problematic”.

Through the above-mentioned process, the balance function and balance recovery of the subject 100 with respect to variation of the visual stimulus are evaluated, and the results are output.

At S108, the evaluation unit 18 may simply determine only whether the visual balance score B calculated after decreasing of the visual stimulus level to Level 0 has recovered to the reference value Bₖ within the predetermined time T2.

As described above, the evaluation unit 18 may determine whether the visual balance score has recovered to the reference value Bₖ within either the first predetermined time T1 or the second predetermined time T2, and vary the output in accordance with the determination result. Specifically, if the observed balance recovery corresponds to the case illustrated in FIG. 8A, for example, the following message may be output:

“The test is finished. You score is 60. Balance recovery is good”.

If the observed balance recovery corresponds to the case illustrated in FIG. 8B, for example, the following message may be output:

“The test is finished. You score is 50. Balance recovery is normal”.

In the exemplary embodiment, the visual stimulus level is sequentially increased from the lowest level, Level 0, to Level 4. In this regard, there are differences in balance function among individual subjects 100. There may be cases where, for a given subject, even a visual stimulus of Level 0 is sufficient, whereas for another subject 100, Level 0 is not sufficient and it is desired to set the lowest level to Level 1.

Accordingly, the balance function of the subject 100 in standing posture with no visual stimulus presented may be evaluated first, and then the visual stimulus level may be varied in accordance with the result.

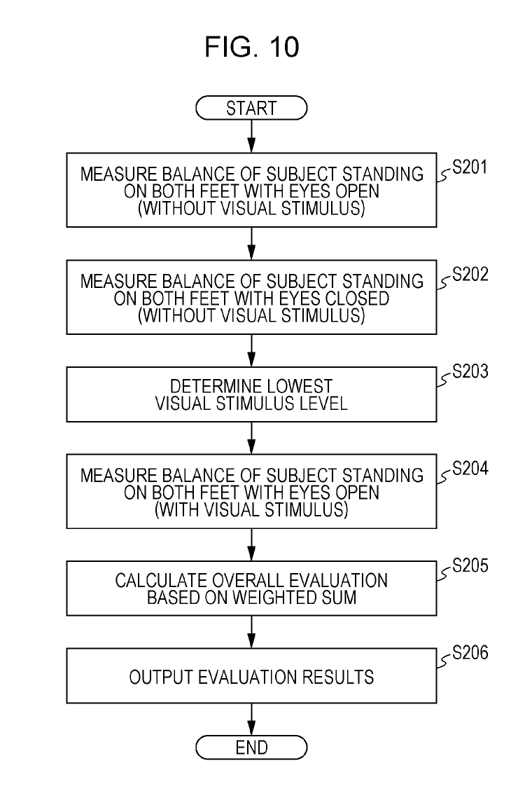

FIG. 10 is an overall process flowchart in this case.

First, the visual pattern control unit 22 performs a control such that no visual pattern is displayed on the screen 13 and thus no visual stimulus is presented. The subject 100 then keeps a standing posture while standing on both feet with the eyes open. The balance measurement unit 16 measures the balance measurement value in this state (S201).

Next, the visual pattern control unit 22 performs a control such that no visual pattern is displayed on the screen 13 and thus no visual stimulus is presented. The subject 100 then keeps a standing posture while standing on both feet with the eyes closed. The balance measurement unit 16 measures the balance measurement value in this state (S202).

The visual pattern control unit 22 uses the balance measurement value calculated at step S201, and the balance measurement value calculated at step S202 to determine the lowest level of the visual stimulus to be presented to the subject 100 (S203). Specifically, if the sum obtained by summing the balance measurement values respectively calculated at steps S201 and S202 is relatively large, and hence the subject's balance function in standing posture with no visual stimulus presented is evaluated to have already decreased, the visual pattern control unit 22 sets the lowest visual stimulus level to a comparatively small value, that is, Level 0. By contrast, if the sum obtained by summing the balance measurement values respectively calculated at steps S201 and S202 is relatively small, and hence the subject's balance function in standing posture with no visual stimulus presented is evaluated to be good, the visual pattern control unit 22 sets the lowest visual stimulus level to a comparatively large value, that is, Level 1.

After determining the lowest visual stimulus level, the visual pattern control unit 22 displays a visual pattern on the screen 13 so that a visual stimulus is presented. The subject 100 then keeps a standing posture while standing on both feet with the eyes open. The balance measurement unit 16 measures the balance measurement value in this state, and the evaluation unit 18 calculates the visual balance score in this state (S204).

After the balance measurement values are calculated at steps S201 and S202 and the visual balance score is calculated at step S204, the balance measurement values and the visual balance score are summed with weights to provide an overall evaluation of the balance function and balance recovery of the subject 100 (S205), and the results are output (S206). Specifically, this is performed as follows. For the balance measurement value calculated by the balance measurement unit 16, a smaller value indicates better balance function, and for the visual balance score calculated by the evaluation unit 18, a greater score indicates better balance function. With these facts taken into account, letting B1 and B2 respectively represent the balance measurement value obtained at step S201 and the balance measurement value obtained at step S202, and B represent the visual balance score obtained at step S204, an overall evaluation is calculated as follows:

overall evaluation=g1/B1+g2/B2+g3·B,

where g1, g2, and g3 are weights.

In FIG. 10, either one of the process at step S201 and the process at step S202 may be omitted, or alternatively, which process is to be executed may be determined for each subject 100. If the process at step S202 is omitted, g2 may be set as g2=0 in calculating an overall evaluation.

FIGS. 11 to 13 illustrate examples of visual patterns each displayed as an image on the screen 13.

FIG. 11 illustrates a concentric visual pattern 13a. By displaying the concentric visual pattern 13a, and varying the rate at which to shake the concentric visual pattern 13a back and forth with respect to the subject 100, the level of the visual stimulus presented may be varied. For example, the visual stimulus level may be varied as follows.

Level 0: visual pattern stopped

Level 1: visual pattern moved back and forth at 0.5 Hz

Level 2: visual pattern moved back and forth at 1.0 Hz

Level 3: visual pattern moved back and forth at 1.5 Hz

Level 4: visual pattern moved back and forth at 2.0 Hz

FIG. 12 illustrates a spiral visual pattern 13b. By displaying the spiral visual pattern 13b, and varying the rate at which to rotate the spiral visual pattern 13b with respect to the subject 100, the level of the visual stimulus presented may be varied. For example, the visual stimulus level may be varied as follows.

Level 0: visual pattern stopped

Level 1: visual pattern rotated in one direction at 5 seconds/revolution

Level 2: visual pattern rotated in one direction at 3 seconds/revolution

Level 3: visual pattern rotated in one direction at 1 second/revolution

Level 4: visual pattern rotated in both directions at 1 second/revolution

In this regard, one direction refers to either the clockwise or counterclockwise direction, and both directions refer to the clockwise and counterclockwise directions.

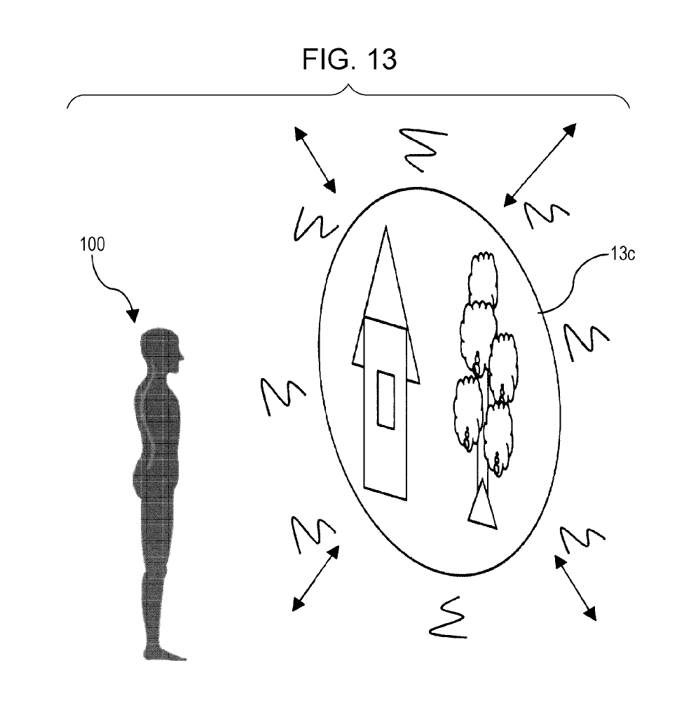

FIG. 13 illustrates a given landscape visual pattern 13c. By displaying the landscape visual pattern 13c, and varying the period and amplitude of vibration of the visual pattern with respect to the subject 100, the level of the visual stimulus presented may be varied as follows.

Level 0: visual pattern stopped

Level 1: vibration magnitude 1 (period: 0.5 Hz, amplitude: very small)

Level 2: vibration magnitude 2 (period: 1.0 Hz, amplitude: small)

Level 3: vibration magnitude 3 (period: 1.5 Hz, amplitude: medium)

Level 4: vibration magnitude 4 (period: 2.0 Hz, amplitude: large)

Although an exemplary embodiment of the present disclosure has been described above, the present disclosure is not limited to the exemplary embodiment but various modifications are possible. Such modifications will be described below.

### Modification 1

In the exemplary embodiment, the evaluation unit 18 calculates a visual balance score as follows:

visual balance score=Σ(visual stimulus level*1/balance measurement value).

In this regard, a visual balance score can be calculated by any formula using the balance measurement value calculated by the balance measurement unit 16. Generally, a visual balance score can be calculated as a function f as follows:

visual balance score=f(visual stimulus level, balance measurement value).

Further, other than the movement distance hd of the head COG position g_{head}, the Lissajous figure area ha, the movement distance fd of the body COG position g_{fp}, and the Lissajous figure area fa, the balance measurement unit 16 may also use values such as the first or second derivative of the head COG position g_{head} and the first or second derivative of the body COG position g_{fp} in calculating a balance measurement value.

### Modification 2

In the exemplary embodiment, the visual stimulus level is varied in accordance with the visual balance score. In this regard, the visual pattern may be varied in accordance with the magnitude relationship between the trajectory length of the head COG position g_{head} and the trajectory length of the body COG position g_{fp}. For example, letting k be a fixed value, if

fd>hd+k,

this may indicate that oscillations in the body COG position g_{fp} are far greater than oscillations in the head COG position g_{head}, and that the leg strength of the subject 100 has declined severely. The visual pattern is thus changed accordingly. By contrast, if

hd>fd+k,

this may indicate that oscillations in the body COG position g_{fp} are far smaller than oscillations in the head COG position g_{head}, and that the visual cognitive function of the subject 100 has declined severely. The visual pattern is thus changed accordingly.

### Modification 3

In the exemplary embodiment, as illustrated in the process flowchart of FIG. 9, the visual balance score of the subject 100 is calculated and evaluated collectively for when the above-mentioned two conditions are met and for when the two conditions are not met. Alternatively, the visual balance score for when the two conditions are met and the visual balance score for when the two conditions are not met may be calculated and evaluated individually. For example, for a given subject, the following visual balance scores are individually calculated for evaluation.

visual balance score for when the two conditions are met=70

visual balance score for when the two conditions are not met=50

If the two conditions are not met, the visual stimulus level is decreased from, for example, Level 4 to Level 0, and the visual balance score is calculated while sequentially increasing the visual stimulus level in accordance with the subsequent recovery of the subject's balance. In this sense, it can be said that the visual balance score for when the two conditions are not met evaluates the balance recovery of the subject 100 more directly.

### Modification 4

In the exemplary embodiment, as illustrated in the process flowchart of FIG. 9, the visual stimulus level is decreased from, for example, Level 4 to the lowest level, Level 0, if the above-mentioned two conditions are not met. Alternatively, instead of decreasing the visual stimulus level to the lowest level, the visual stimulus level may be decreased one level from the current level, or may be decreased two levels from the current level. The number of levels to be decreased at this time may be determined in accordance with the characteristics of the subject 100. For example, the number of levels to be decreased may be set to vary with the age of the subject 100.

### Modification 5

In the exemplary embodiment, the subject 100 is presented with a visual stimulus while standing on both feet with the eyes open. Instead of or in addition to this, the subject 100 may be presented with a visual stimulus while standing on one foot with the eyes open. For example, in calculating the visual balance score for the subject 100 with good balance function as illustrated in FIG. 8A, a test executed with the subject 100 standing on both feet with the eyes open may be followed by a test executed with the subject 100 standing on one feet with the eyes open.

The foregoing description of the exemplary embodiment of the present disclosure has been provided for the purposes of illustration and description. It is not intended to be exhaustive or to limit the disclosure to the precise forms disclosed. Obviously, many modifications and variations will be apparent to practitioners skilled in the art. The embodiment was chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A recovery evaluation apparatus comprising:
a display that displays an image;
an evaluating unit that evaluates balance of a subject who is viewing the image;
a controller that, in accordance with temporal variation of an evaluation value of the balance, varies a degree of a visual stimulus provided by the image; and
a recovery evaluation unit that evaluates balance recovery, the balance recovery representing balance recovery of the subject upon varying of the degree of the visual stimulus.

2. The recovery evaluation apparatus according to claim 1,
wherein the controller sequentially increases the degree of the visual stimulus if a degree of the temporal variation of the evaluation value is less than a threshold.

3. The recovery evaluation apparatus according to claim 2,
wherein the controller decreases the degree of the visual stimulus when the evaluation value exceeds an allowable value as the controller sequentially increases the degree of the visual stimulus.

4. The recovery evaluation apparatus according to claim 2,
wherein the controller decreases the degree of the visual stimulus when an abnormality occurs in the subject as the controller sequentially increases the degree of the visual stimulus.

5. The recovery evaluation apparatus according to claim 1,
wherein the controller sequentially increases the degree of the visual stimulus if a degree of the temporal variation of the evaluation value is less than a threshold and if the evaluation value is less than an allowable value, and
wherein the controller decreases the degree of the visual stimulus if the degree of the temporal variation of the evaluation value exceeds the threshold or if the evaluation value exceeds the allowable value.

6. The recovery evaluation apparatus according to claim 3,
wherein the controller sequentially increases the degree of the visual stimulus if the evaluation value recovers to a reference value within a predetermined time after the controller decreases the degree of the visual stimulus.

7. The recovery evaluation apparatus according to claim 3,
wherein the controller stops evaluation performed by the recovery evaluation unit if the evaluation value does not recover to a reference value within a predetermined after the controller decreases the degree of the visual stimulus.

8. The recovery evaluation apparatus according to claim 1,
wherein the recovery evaluation unit evaluates the balance recovery of the subject by further using at least one of a first balance evaluation value and a second balance evaluation value, the first balance evaluation value representing a value of balance evaluated with the subject opening eyes and not viewing the image, the second balance evaluation value representing a value of balance evaluated with the subject closing eyes.

9. The information processing apparatus according to claim 8,
wherein the controller varies the degree of the visual stimulus by use of at least one of the first balance evaluation value and the second balance evaluation value.

10. The recovery evaluation apparatus according to claim 1,
wherein the recovery evaluation unit evaluates the balance recovery by use of a trajectory of a center of gravity of a head of the subject and a trajectory of a center of gravity of a foot pressure of the subject.

11. A non-transitory computer readable medium storing a program causing a computer to execute a process, the process comprising
displaying an image on a screen;
evaluating balance of a subject who is viewing the image;
in accordance with temporal variation of an evaluation value of the balance, varying a degree of a visual stimulus provided by the image; and
evaluating balance recovery, the balance recovery representing balance recovery of the subject upon varying of the degree of the visual stimulus.

12. A recovery evaluation apparatus comprising:
display means for displaying an image;
evaluating means for evaluating balance of a subject who is viewing the image;
control means for, in accordance with temporal variation of an evaluation value of the balance, varying a degree of a visual stimulus provided by the image; and
recovery evaluation means for evaluating balance recovery, the balance recovery representing balance recovery of the subject upon varying of the degree of the visual stimulus.
